(19) United States
(12) Patent Application Publication
(10) Pub. No.: US 2020/0156066 A1
(43) Pub. Date: May 21, 2020

(54) MICROFLUIDIC DEVICE AND APPARATUS

(71) Applicant: EPIGEM LIMITED, Redcar Cleveland (GB)

(72) Inventors: Philip SUMMERSGILL, Redcar Cleveland (GB); Simon ALLEN, Yarm Yorkshire (GB); Timothy George RYAN, Middlesborough Cleveland (GB); Niamh Aine KILCAWLEY, Beaumont, Dublin (IE)

(21) Appl. No.: 16/604,400

(22) PCT Filed: Apr. 16, 2018
(86) PCT No.: PCT/GB2018/050992

§ 371 Date: Oct. 10, 2019

(30) Foreign Application Priority Data
Apr. 26, 2017 (GB) 1706616.8

(51) Int. Cl. B01L 3/00; C12Q 1/02

(52) U.S. Cl. CPC B01L 3/502715; B01L 3/50273; B01L 3/502761; C12Q 1/02; B01L 2200/0647; B01L 2400/049; B01L 2300/06; B01L 2300/0627; B01L 2300/0883; B01L 2300/047; B01L 2200/16

(57) ABSTRACT

A microfluidic test apparatus including a microfluidic device having a first reservoir for receiving a first fluid containing a sample of cells, a microfluidic test region, a first microfluidic pathway provided between the microfluidic test region and the first reservoir; and a port for connection to a pump, the apparatus including a first pump connected to the port and configured to pump a priming fluid into the port, a second pump connected to the port and configured to apply suction at the port when operated and a controller configured to control operation of the first and second pumps, where the controller operates the first pump to prime the microfluidic device and operates the second pump to draw a test volume from the first reservoir into the microfluidic test region.

### TECHNICAL FIELD

The present disclosure relates to a microfluidic device and to a microfluidic test apparatus. The microfluidic device and microfluidic test apparatus have particular utility in performing tests on fluid samples of cells.

### BACKGROUND

Microfluidic devices are devices with very small features, typically in the μm range, which perform operations on very small fluid samples, typically in the μl range. The small volume of fluid required for use with a microfluidic device offers benefits in fields such as medicine, since only a very small blood sample is needed.

One such application of microfluidic devices is described by Lei Li et al. in “A microfluidic platform for osmotic fragility test of red blood cells”, RSC Advances, 2012, 2, 7161-7165. Li describes the use of two syringe pumps to push a blood sample and pure water into a microfluidic device. The microfluidic device of Li consists of a Y junction at which the blood sample and pure water meet and form a laminar flow and a length of serpentine channel consisting of 40 square-wave structures. The blood sample and pure water pass along the channel and then exit the microfluidic device at a waste outlet. In the device of Li, the fragility of red blood cells are tested along the length of the channel. An image capture device captures images of the blood sample at several places along the channel, and these images are analysed to determine an osmotic fragility curve from the number of blood cells present at each place along the channel.

### SUMMARY OF THE DISCLOSURE

In a first aspect of the present disclosure, a microfluidic test apparatus is provided, comprising a microfluidic device. The microfluidic device comprises a first reservoir for receiving a first fluid containing a sample of cells, a microfluidic test region, a first microfluidic pathway provided between the microfluidic test region and the first reservoir; a port. The microfluidic test apparatus further comprises a first pump connected to the port and configured to pump a priming fluid into the port, and a second pump connected to the port and configured to apply suction at the port when operated. A controller is provided, which is configured to control operation of the first and second pumps, wherein the controller operates the first pump to prime the microfluidic device and operates the second pump to draw a test volume from the first reservoir into the microfluidic test region.

In a second aspect of the present disclosure, a microfluidic device is provided, comprising a first reservoir for receiving a first fluid comprising a sample of cells and a microfluidic test region. A first microfluidic pathway is provided between the microfluidic test region and the first reservoir. The microfluidic device further comprises a port for connection to a pump, the pump in use applying suction at the port to draw a test volume from the first reservoir into the microfluidic test region. A microfluidic waste region is provided between the microfluidic test region and the port, wherein the microfluidic waste region defines a microfluidic volume commensurate with the test volume.

Other features and aspects of this disclosure will be apparent from the following description and the accompanying drawings.

### BRIEF DESCRIPTION OF THE DRAWINGS

FIG. 1 is a schematic diagram of a microfluidic test apparatus according to one embodiment of the present disclosure;

FIGS. 2a, 2b, and 2c are schematic diagrams of microfluidic devices according to embodiments of the present disclosure;

FIG. 3 is an enlarged view of a microfluidic test region from a microfluidic device according to one embodiment of the present disclosure;

FIGS. 4a and 4b are images of red blood cells in a microfluidic test region from a microfluidic test apparatus for blood samples that are normal and that have sickle-cell disease, respectively;

FIGS. 5a and 5b are images of red blood cells in a microfluidic test region from a microfluidic test apparatus for blood samples that are normal and that have sickle-cell disease, respectively;

FIGS. 6a and 6b are images of red blood cells in a microfluidic test region from a microfluidic test apparatus for blood samples that are normal and that have hereditary spherocytosis, respectively;

FIG. 7 shows a red blood cell profile along a microfluidic test region from the test shown in FIGS. 6a and 6b.

FIG. 8 shows, schematically, a microfluidic test apparatus according to an embodiment of the disclosure; and

FIGS. 9A-9C illustrate component parts of an embodiment of a microfluidic device of the disclosure.

### DETAILED DESCRIPTION

Reference will now be made in detail to specific embodiments or features, examples of which are illustrated in the accompanying drawings. Wherever possible, corresponding or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

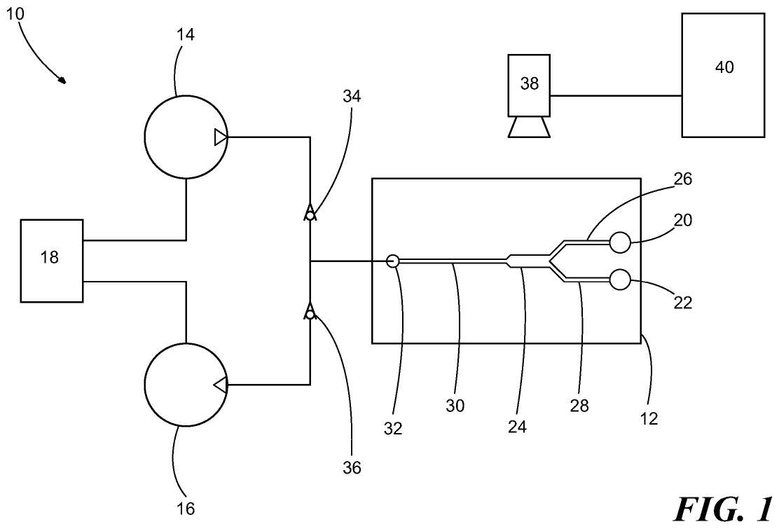

FIG. 1 is an illustrative schematic view of a microfluidic test apparatus 10 according to embodiments of the present disclosure. The microfluidic test apparatus 10 comprises a microfluidic device 12, first and second pumps 14 and 16, respectively, and a controller 18 that operates the pumps 14, 16.

The microfluidic device 12 comprises a first reservoir 20 and a second reservoir 22 for receiving a first fluid and a second fluid, respectively, and a microfluidic test region 24. A first microfluidic pathway 26 is provided between the first reservoir 20 and the microfluidic test region 24. A second microfluidic pathway 28 is provided between the second reservoir 22 and the microfluidic test region 24. In the microfluidic device 12 illustrated in FIG. 1, a further microfluidic pathway 30 is provided between the microfluidic test region 24 and a port 32.

The first pump 14 is connected to the port 32 via a valve 34. The first pump 14 and valve 34 are arranged to pump a priming fluid into the port 32 when operated. The first pump 14 may be a syringe pump, in which the syringe filled with the priming fluid. The priming fluid may contain a wetting agent to reduce air being trapped in the microfluidic device 12.

The second pump 16 is connected to the port 32 via a valve 36. The second pump 16 and valve 36 are arranged to apply suction at the port 32 when operated and draw fluid therefrom. The valves 34 and 36 may take any suitable form, including a one-way valve, non-return valve, or an activated valve. In some embodiments the valves 34, 36 may be omitted.

The controller 18 is configured to control operation of the first and second pumps 14 and 16, and the valves 34, 36 where the valves are activated. The controller 18 may be any suitable device such as a microcontroller, embedded controller, programmable logic controller (PLC), microprocessor, portable computing device or computer and may include a control program. The controller 18 is configured to operate the first pump 14 to prime the microfluidic device 12. The first pump 14 preferably has a pump rate in the order of mL/second (e.g. 1-10 mL/s), and preferably mL/minute (e.g. in the range of 1-10, or 1-100, or 1-200 mL/min); this relatively high flow rate aids priming the microfluidic device 12 and reduces or eliminates air entrapment. The controller 18 operates the first pump 14 to pump priming fluid into the microfluidic device 12 such that priming fluid enters the reservoirs 20, 22.

After priming, the first and second fluids are then added to the reservoirs 20 and 22, respectively. Where priming fluid has entered the reservoirs 20, 22, in some embodiments the priming fluid may be removed before the first and second fluids are added. The first fluid comprises a sample of cells. The second fluid is chosen according to the test requirements and may for example include a label and/or a stressor to the cells that cause a distinctive change in cells which may include cell lysis, aggregation, swelling, shrinkage, and/or shape change. In some embodiments, a series of second fluids may be added one by one to the second reservoir 22 as a test is performed, each second fluid having a different stressors, stressor concentration, and/or different labels. One example of a suitable label/dye is eosin-5-maleimide (EMA), which may be used for instance to stain band 3 proteins following shear stress.

The controller 18 is configured to operate the second pump 16 to draw a test volume of first fluid from the first reservoir 20 into the microfluidic test region 24. A volume of second fluid will also be drawn from the second reservoir 22, according to the dimensions of the microfluidic pathways 26, 28. Since the second pump 16 applies suction to the port 32, pressure on the cells in the first fluid is limited. Using a pump to ‘push’ the first fluid through the microfluidic device 12 can result in higher pressure on the cells and cause cell ruptures, which may affect testing. It is preferred that the second pump 16 has a pump rate in the order of μL/minute (e.g. 10-100, or 10-200, or 10-500 μL/m) or μL/second (e.g. 1-10 μL/s, or 1-100 μL/s or 1-500 μL/s).

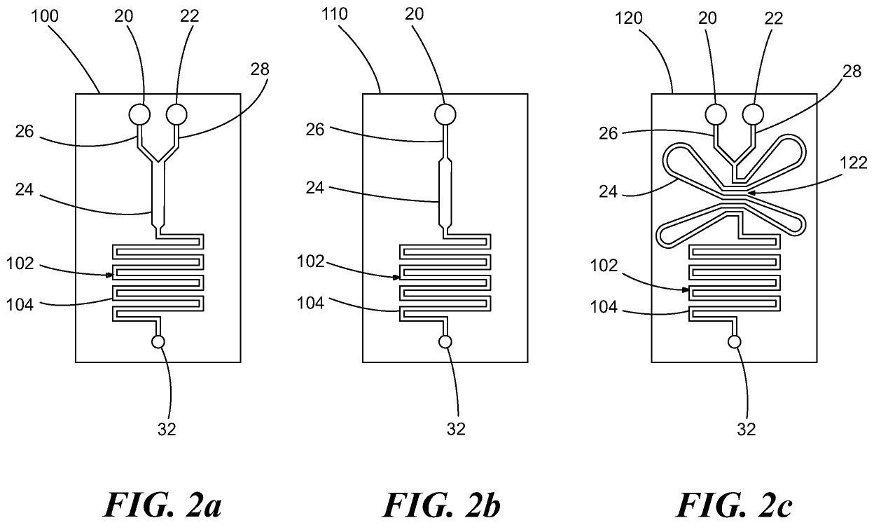

In the microfluidic device 12 shown in FIG. 1, the microfluidic test region 24 comprises a microfluidic channel into which the first and second fluids flow. Other forms of microfluidic test region 24 may be employed; for instance, the microfluidic test region 24 may comprise a microfluidic channel formed into a spiral. Forming the microfluidic test region 24 in a spiral may permit the imaging device 38 to capture fluid flow at several locations along the microfluidic test region 24 in a small area covered by a single image. Other configurations of the microfluidic test region 24 are possible, one example of which is described below in relation to FIG. 2c. The dimensions of the microfluidic channel may be determined according to requirements, such as desired fluid flow rate, and test sample volume.

The microfluidic test apparatus 10 further comprises a sensor responsive to the microfluidic test region 24. The sensor may be any form of sensor according to the test being performed. In the embodiment illustrated in FIG. 1, the sensor comprises an imaging device 38. The imaging device 38 captures images of the microfluidic test region 24 as the first and second fluids pass along it. In some embodiments, a region (not shown) of colour filter may be provided in the microfluidic test device 12 above the microfluidic test region 24, which may improve contrast in the images captured by the imaging device 38. A dye or marker may also be used, such as a fluorescent or chemiluminescent dye or marker. The test region may be configured for multiparameter testing to identify cell related differences and also serum related, including serology testing using antigen/antibody binding using a printed panel of antigens in the test region to recognise sought proteins in the serum or in the cell surface. Suitable surface chemistries may be used to prevent surface adhesion except in targeted areas in the sensor area of the test region by spotting or printing with specific molecular moieties for targeted molecular trapping or binding.

The microfluidic test apparatus 10 further comprises an image processor 40 that analyses images received from the imaging device 38. The image processor 40 may be configured to perform one or more forms of analysis of images received form the imaging device 38. Such analysis may include cell counts at locations along the microfluidic test region 24, cell counts at one or more locations in the microfluidic test region 24 which may have an affinity substance applied thereto, cell shape, to name a few. Where the second fluid is a stressor to the cells, the image processor 40 may be configured to determine a cell lysis or cell shape change profile across the microfluidic test region 24, and may also be configured to compare or display the cell lysis or shape change profile to one or more control profiles. The imaging device 38 can also be used as part of a control system to ensure that the rate of movement of cells within the test region 24 is kept constant for each test, such that the residence time in the test region 24 is monitored and controlled by control of pump 16.

Referring now to FIG. 2a, a microfluidic device 100 according to another embodiment of the present disclosure is shown. The microfluidic device 100 is similar to the microfluidic device 12 shown in FIG. 1, with like reference numerals denoting like parts. The microfluidic device 100 differs from the microfluidic device 12 in that the microfluidic device 100 is provided with a microfluidic waste region 102 provided between the microfluidic test region 24 and the port 32. The microfluidic waste region 102 may comprise a circuitous microfluidic pathway 104. While shown in two dimensions in the drawings for clarity it will be appreciated that the pathway 104 may be formed in three dimensions. The microfluidic waste region 102 defines a microfluidic volume commensurate with the test volume to prevent the first or second fluids from reaching the port 32. The microfluidic waste region 102 prevents the first or second fluid from leaving the microfluidic device 100, thereby avoiding cross-contamination that would result if some of the first or second fluids were to leave the microfluidic device 12 and then subsequently be pumped into another microfluidic device during the priming thereof.

FIG. 2b shows a microfluidic device 110 according to a further embodiment of the present disclosure. The microfluidic device 110 is similar to the microfluidic device 100 shown in FIG. 2a with like reference numerals denoting like parts. The microfluidic device 110 differs from the microfluidic device 100 in that the microfluidic device 110 omits the second reservoir 22 and second microfluidic pathway 28. The microfluidic device 110 may be used where a stressor has been added to the first sample or where mechanical stress is applied in the microfluidic device.

FIG. 2c shows a microfluidic device 120 according to a further embodiment of the present disclosure. The microfluidic device 120 is similar to the microfluidic device 100 shown in FIG. 2a with like reference numerals denoting like parts. The microfluidic device 120 differs from the microfluidic device 100 in the configuration of the microfluidic test region 24. The microfluidic test region 24 of the microfluidic device 120 comprises a serpentine channel which passes back and forth through a central region 122. The central region 122 provides a compact area that can be imaged by the imaging device 38 to capture information at several locations along the microfluidic test region 24 without requiring several imaging sensors.

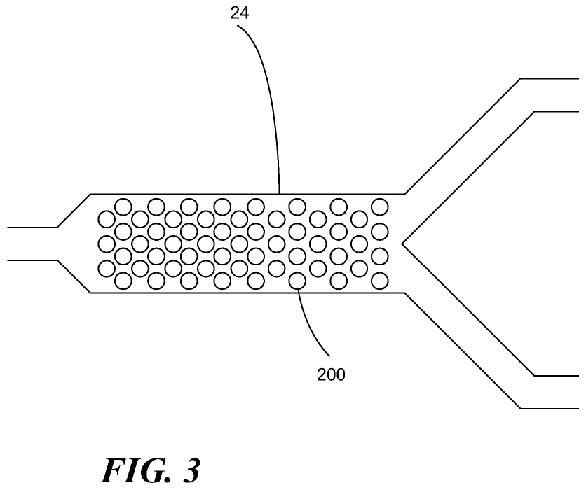

Referring now to FIG. 3, a microfluidic test region 24 from a microfluidic device 12 or 100 according to further embodiments of the invention is shown. The microfluidic test region 24 has a plurality of obstacles 200 formed therein. It will be appreciated that the size, shape, quantity and density of the obstacles 200 may be varied from what is shown, and further that the size, shape, quantity and density of the obstacles 200 may be varied along the microfluidic test region 24. In some embodiments of the disclosure, a first affinity substance is formed on at least one of the obstacles 200. In other embodiments of the present disclosure, a plurality of affinity substances are provided, each affinity substance being formed on a group of obstacles 200 associated therewith. The image processor 40 may then count cell affinity to obstacles or groups of obstacles to which an affinity substance has been applied. Any suitable affinity substances known to those skilled in the art may be used, including cationic or anionic polymers. Diffusive mixing under laminar flow conditions or mixing geometries that induce turbulent mixing may be used in the microfluidic test region 24.

It will be appreciated that the foregoing examples of microfluidic devices are exemplary only, and that further configurations are possible according to test requirements. For instance, in some embodiments, more than one microfluidic test region may be provided, more than two reservoirs may be used. In other embodiments, the second microfluidic pathway 28 may include a junction to split into two pathways that sandwich the first microfluidic pathway 26, one to either side, so that the first fluid has the second fluid on both side in the microfluidic test region.

### EXAMPLES

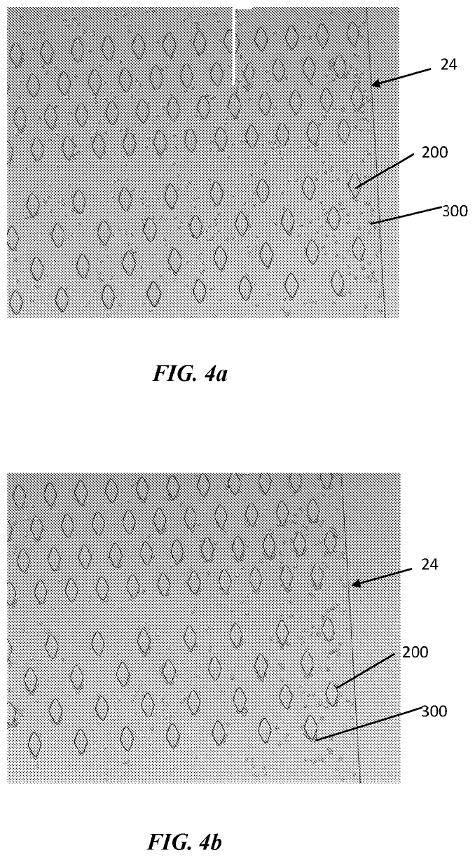

FIGS. 4a and 4b are images from the image sensor 38 of a microfluidic test region 24 in which obstacles 200 are present which provide mechanical stress to cells passing through the test region 24. The direction of fluid flow in FIGS. 4a and 4b is from the bottom of the image to the top of the image. FIG. 4a is an image showing red blood cells 300 from a healthy patient. FIG. 4b is an image showing red blood cells (RBC) 300 from a patient with sickle-cell disease. As can be seen, the RBC 300 in the patient in FIG. 4b have an increased tendency to adhere to the obstacles 200. A microfluidic device, such as that shown in FIG. 2b, with a single reservoir was used for the tests shown in FIGS. 4a and 4b since stress was provided mechanically.

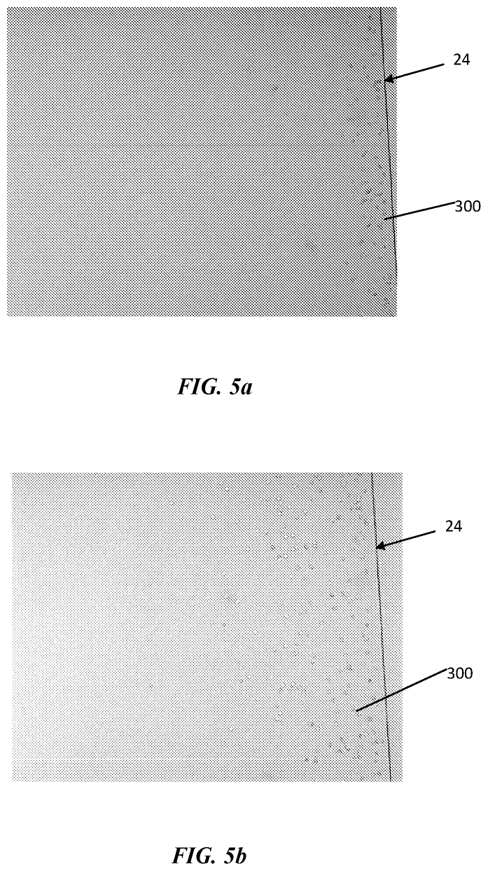

FIGS. 5a and 5b are images from the image sensor 38 of a microfluidic test region 24. The second fluid used in this example was a stressor in the form of dilute HCl at 0.5% concentration by volume in buffer. As the first fluid containing red blood cells and the second fluid containing the dilute HCl enter the microfluidic test region 24, a laminar flow results, with the HCl diffusing into the first fluid. Since the fluids are flowing along the microfluidic test region, a diffusion gradient forms along the length of the microfluidic test region 24. In FIGS. 5a and 5b the HCl has diffused from left to right. The images in FIGS. 5a and 5b are taken at the same point along the microfluidic test region 24. As would be appreciated, images may be take at several locations along the microfluidic test region 24 by the imaging device 38, and the image processor 40 may then count RBC in each image to produce a lysis profile as the HCl diffuses and stresses the RBC. FIG. 5a is an image showing red blood cells 300 from a healthy patient. FIG. 5b is an image showing red blood cells (RBC) 300 from a patient with sickle-cell disease. As can be seen, RBC in patients with sickle-cell disease are more resistant to lysing from the HCl.

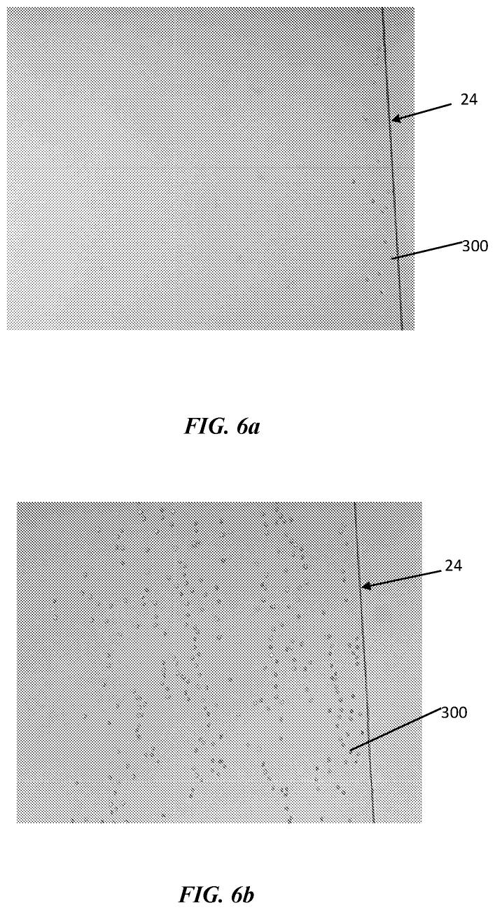

FIGS. 6a and 6b are images from the image sensor 38 of a microfluidic test region 24. The second fluid used in this example was a stressor in the form of dilute HCl at 0.5% concentration by volume in buffer. FIG. 6a is an image showing red blood cells 300 from a healthy (control) patient. FIG. 6b is an image showing red blood cells (RBC) 300 from a patient with hereditary spherocytosis. As can be seen, RBC in patients with hereditary spherocytosis are more resistant to lysing from the HCl.

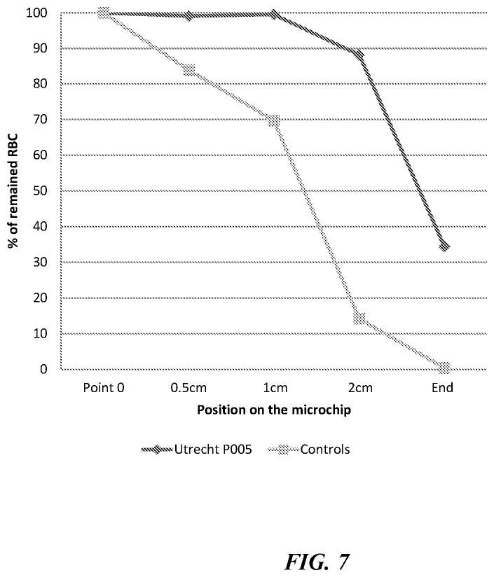

FIG. 7 is a profile of RBC count from the test shown in FIGS. 6a and 6b, as a percentage of a RBC count from an image taken at the start of the microfluidic test region 24. Further images were taken at 0.5 cm, 1 cm, 2 cm and the end of the microfluidic test region 24. In FIG. 7, the curve labelled ‘control’ represents a RBC count from a healthy patient, while the curve labelled ‘Utrecht P005’ represents a RBC count from a patient with hereditary spherocytosis. As can be seen from FIG. 7, the profile of RBC count along the microfluidic test region 24 is markedly different.

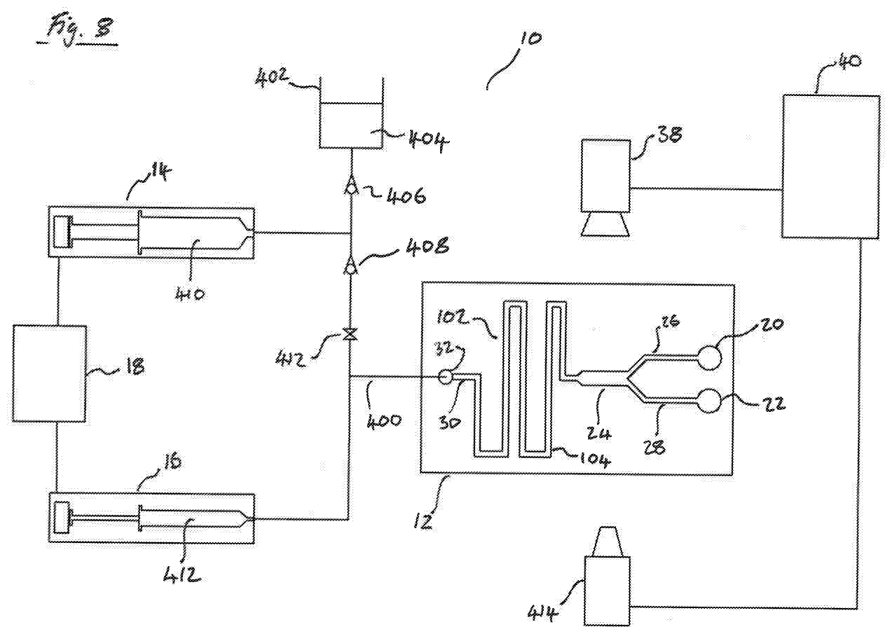

FIG. 8 illustrates a further preferred embodiment of a microfluidic test apparatus, generally indicated by 10. The test apparatus comprises a microfluidic device 12 having first 20 and second 22 reservoirs for receiving a first and second fluid, respectively, a microfluidic test region 24 and a waste region 102, preferably microfluidic, said waste region 102 comprising a circuitous pathway 104. Again, a first microfluidic pathway 26 is provided between the first reservoir 20 and the test region 24, and a second microfluidic pathway 28 is provided between the second reservoir 22 and the test region 24.

A microfluidic pathway 30 is also provided between the waste region 102 and the port 32. In use, the port 32 is connectable to a fluid pathway 400 connecting the microfluidic device 12 to two pumps 14, 16, and priming reservoir 402 for holding priming fluid. The outlet of the reservoir 402 is provided with a valve 406 to allow priming fluid to leave the priming reservoir, but not to return. This may be achieved by use of a one-way valve, non-return valve, or an isolation valve, preferably controlled in tandem with the pumps 14, 16. A second such valve 408 may also be provided in the fluid pathway, with a connection to the pump 14 provided between the two valves 406, 408. In this preferred embodiment, the pump 14 is a syringe pump, of relatively large volume, e.g. at least as large as the total fluid volume of the microfluidic device 12 and the interconnecting fluid pathway 400. The pump may be activated in a first mode to draw priming fluid 404 into the barrel 410 of the syringe pump 14. The two valves 406, 408 operate to ensure that the flow is from priming reservoir 402, rather than from any connected microfluidic device 12.

The pump 14 may then be operated in a second mode to push priming fluid through the fluid pathway, into the microfluidic device 12 and eventually into the reservoirs 20, 22 as described above. A further isolation valve 412 may also be provided, either manually-operated, or controlled in tandem with the pump controls, to enable the priming fluid reservoir to be isolated from the microfluidic device and the second pump 16. The priming reservoir 402 may be provided with a level sensor (not illustrated) to monitor the amount of priming fluid 404 available, and to raise a user alarm if more fluid 404 needs to be added.

Once the microfluidic device 12 has been primed, a sample (e.g. of cells, especially red blood cells) may be added to one of the reservoirs 20, and a reagent (e.g. a stressor, or marker dye) may be added to the other reservoir 22. The second pump 16 may then be activated to draw fluid through the microfluidic device 12, as described above, for analysis. In this preferred embodiment, the second pump 16 is also a syringe pump, and is preferably configured such that the volume of its barrel 412 is less than the volume of the microfluidic waste region 102 of the microfluidic device 12. This ensures that neither the fluid pathway 200 nor the pump 16 can be contaminated with any material introduced into the microfluidic device 12.

As described above, the apparatus 10 also includes a controller, to control at least the operation of the pumps. The apparatus also includes an imaging device and an imaging processor 40. For some applications, a fluorescent marker might be used in the analysis, and in this instance an illuminator 414 may be provided to illuminate the test area 24 with e.g. ultraviolet light.

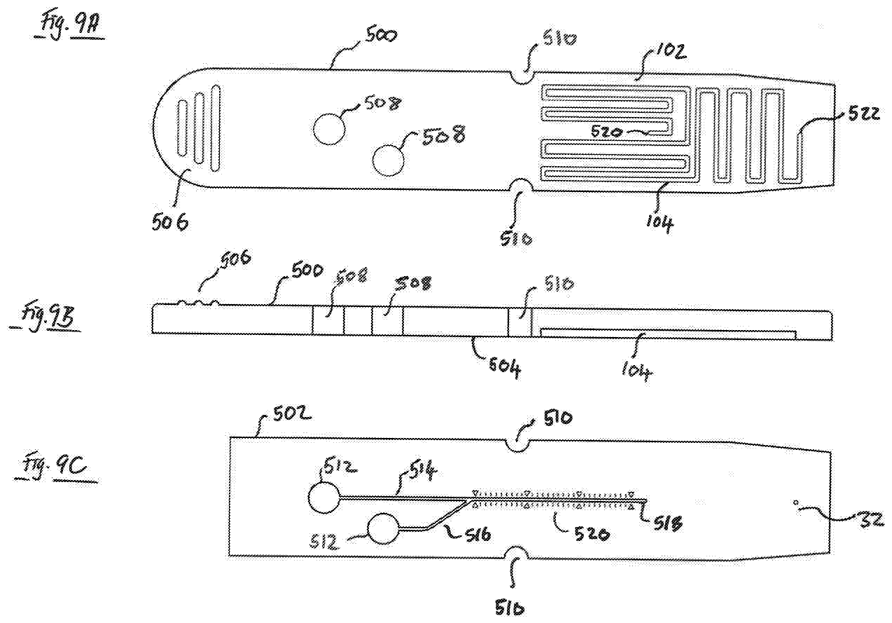

FIGS. 9A-9C illustrate component parts of a preferred microfluidic device 12 of the invention. The device 12 comprises an upper portion 500 and a lower portion 502. In use, the two portions are joined together, e.g. with a thermoplastic adhesive, to form the microfluidic device. The upper portion 500, which may conveniently be made of a material such as plastics, e.g. acrylic or polymethyl methacrylate is shown in plan and elevation view in FIGS. 9A and 9B respectively. Two through-holes 508 are provided, forming the first and second reservoirs 20, 22 when the upper and lower portions are joined together. The upper portion 500 has a waste region 102 formed as a circuitous pathway, preferably a microfluidic pathway. The pathway comprises a continuous channel in the lower surface 504 of the upper portion. When the two portions are joined, the channel is sealed by the upper face of the lower portion 502 forming the pathway. A gripping portion 506 may also be provided, in the form e.g. of raised ribs or indentations, to allow a user to firmly hold the device for positioning in a test apparatus. Indentations 508 may also be provided on each edge of the device 12 to allow it to be positioned in a test apparatus relative to cooperating pins (not illustrated). The indentation may be formed by e.g. moulding, machining, etching or other such method.

The lower portion 502 of the device is illustrated in plan view in FIG. 9C. This portion comprises the accurately-formed microfluidic flow paths, as described above, and may be most conveniently produced by e.g. photo-resist techniques, micro-machining or other such technique known in the art. The flow paths are again in the form of channels, or indentations, in the upper surface of the lower portion 502 which, when abutted to the upper portion 500 form a fluid-tight microfluidic pathway.

Two recessed circular regions 512 are provided that are positioned to interact with the through-holes in the upper region to form the reservoirs 20, 22. First and second recessed channels 514, 516 are provided in fluid communication with each respective recessed circular regions 512 that, when covered by the upper portion 500, form the first and second microfluidic pathways 26, 28 described above. A third recessed channel 518 is also provided, which, when covered by the upper portion 500, forms the microfluidic test region. The third recessed channel 518 is in fluid communication with both the first and second recessed channels 514, 516 to allow two fluids therein to come into contact when the device is used as described herein. In a particularly preferred embodiment illustrated in FIG. 9C, the first recessed channel 514 is co-linear with the third recessed channel 518. In this way, if cells are put into the reservoir in fluid connection with the first microfluidic pathway 514, their flow path is essentially linear. The inventor has found that this reduces unwanted mechanical damage to the cells when the device is in use. It is particularly also preferred that the transverse cross-sectional area of the third recessed channel 518 is equal to the sum of the transverse cross-sectional areas of the first and second recessed channels 514, 516. In this way, the fluid is not subjected to any acceleration when the two fluid streams meet, which might otherwise cause unwanted damage to cells under analysis. Such a feature is preferred for any microfluidic devices described herein.

Indicia 520 may also be provided adjacent the third recessed channel 518 to aid positioning and to provide a reference for the image analysis.

The end of the third recessed channel 518 is positioned such that it fluidly communicates with the proximal end 520 of the waste region. The distal end 522 of the waste region is positioned such that it fluidly communicates with a port 32 (e.g. a through-hole) in the lower portion 502 of the device.

It should be appreciated that the present disclosure is not limited to the foregoing examples. For instance, other stressors may be used, including stressors which induce shrinkage or oxidative stress in RBCs. Based on preliminary test results, the microfluidic test apparatus 10 of the present disclosure may be a useful tool for diagnosis of a rare anaemias and other blood diseases, severity diagnosis, and assessment of the efficacy of treatment. Other tests may also be performed, including a rapid ‘shrinkage’ test for overhydrated RBCs, oxidation resistance tests, RBC membrane surface tests. The test apparatus 10 can be readily programmed for a simple or complex set of assay operations.

Various embodiments disclosed herein are to be taken in the illustrative and explanatory sense, and should in no way be construed as limiting of the present disclosure.

1. A microfluidic test apparatus, comprising:
a microfluidic device comprising:
a first reservoir for receiving a first fluid containing a sample of cells;
a microfluidic test region;
a first microfluidic pathway provided between the microfluidic test region and the first reservoir; and
a port for connection to a pump;
a first pump connected to the port and configured to pump a priming fluid into the port;
a second pump connected to the port and configured to apply suction at the port when operated; and
a controller configured to control operation of the first and second pumps, wherein the controller operates the first pump to prime the microfluidic device and operates the second pump to draw a test volume from the first reservoir into the microfluidic test region.

2. The microfluidic test apparatus of claim 1, further comprising a sensor responsive to the microfluidic test region.

3. The microfluidic test apparatus of claim 2, wherein the sensor comprises an imaging device.

4. The microfluidic test apparatus of claim 3, further comprising an image processor that analyses images received from the imaging device.

5. The microfluidic test apparatus of claim 4, wherein the image processor is configured to determine a cell shape change profile across the microfluidic test region.

6. The microfluidic test apparatus of claim 4, wherein the image processor is configured to determine a count of cell affinity to one or more obstacles provided in the microfluidic test region.

7. The microfluidic test apparatus of claim 6, wherein the image processor is configured to determine a count of cell affinity to one or more groups of obstacles or printed spots provided in the microfluidic test region.

8. The microfluidic test apparatus of claim 1, wherein the microfluidic device further comprises a microfluidic waste region provided between the microfluidic test region and the port, wherein the microfluidic waste region defines a microfluidic volume commensurate with the test volume.

9. The microfluidic test apparatus of claim 1, wherein the microfluidic device further comprises a second reservoir for receiving a second fluid.

10. The microfluidic test apparatus of claim 9, wherein the second fluid is a stressor to the cells.

11. A microfluidic device, comprising:
a first reservoir for receiving a first fluid comprising a sample of cells;
a microfluidic test region;
first microfluidic pathway provided between the microfluidic test region and the first reservoirs;
a port for connection to a pump, the pump in use applying suction at the port to draw a test volume from the first reservoir into the microfluidic test region;
a microfluidic waste region provided between the microfluidic test region and the port, wherein the microfluidic waste region defines a microfluidic volume commensurate with the test volume.

12. The microfluidic device of claim 11, further comprising a second reservoir for receiving a second fluid, and a second microfluidic pathway provided between the microfluidic test region and the second reservoirs.

13. The microfluidic device of claim 11, wherein the microfluidic test region comprises a microfluidic channel.

14. The microfluidic device of claim 13, wherein a plurality of obstacles are provided in the microfluidic channel.

15. The microfluidic device of claim 14, wherein a density of the obstacles varies along the microfluidic channel.

16. The microfluidic device of claim 14, wherein a first affinity substance is formed on at least one of the obstacles.

17. The microfluidic device of claim 14, wherein a plurality of affinity substances are provided, each affinity substance being formed on a group of obstacles associated therewith.

18. The microfluidic device of claim 11, wherein the microfluidic waste region comprises a circuitous microfluidic pathway.
